(19) United States
(12) Patent Application Publication
(10) Pub. No.: US 2023/0270772 A1
(43) Pub. Date: Aug. 31, 2023

(54) USE OF 5'-METHYLTHIOADENOSINE IN PREPARATION OF OBESITY-SUPPRESSING DRUGS OR HEALTH CARE PRODUCTS

(71) Applicant: Zhejiang Chinese Medical University, Hangzhou (CN)

(72) Inventors: Gang Cao, Hangzhou (CN); Qiang Lv, Hangzhou (CN)

(21) Appl. No.: 17/984,855

(22) Filed: Nov. 10, 2022

(30) Foreign Application Priority Data
Feb. 25, 2022 (CN) 202210183725.3

(51) Int. Cl. A61K 31/7076; A61P 3/04

(52) U.S. Cl. CPC A61K 31/7076; A61P 3/04

(57) ABSTRACT

Disclosed is a use of 5′-methylthioadenosine in preparation of obesity-suppressing drugs or health care products. It is demonstrated from animal experiments that the 5′-methylthioadenosine has a significant suppression effect on obesity and has an excellent application prospect in preparation of obesity-suppressing drugs or health care products.

### CROSS REFERENCE TO RELATED APPLICATION

This patent application claims the benefit and priority of Chinese Patent Application No. 202210183725.3, entitled “USE OF 5′-METHYLTHIOADENOSINE IN PREPARATION OF OBESITY-SUPPRESSING DRUGS OR HEALTH CARE PRODUCTS” filed on Feb. 25, 2022, the disclosure of which is incorporated by reference herein in its entirety as part of the present application.

### TECHNICAL FIELD

The present disclosure relates to use of 5′-methylthioadenosine in preparation of obesity-suppressing drugs or health care products, belonging to the technical field of medicine.

### BACKGROUND ART

Obesity is a chronic metabolic disease characterized by abnormal or excessive accumulation of fat in the body, and it is also a predisposing factor for a plurality of other diseases, which will increase the risk of type II diabetes, hypertension, cancer and other diseases. With the rapid economic development and changes in people's lifestyles, the incidence of obesity in the global population has increased significantly, becoming an increasingly serious global health problem. According to the statistics of the World Health Organization (WHO), nearly 2.3 billion adults and children worldwide were overweight (BMI≥25 kg/m²) in 2019 (Sung et al. CA: a cancer journal for clinicians, 2019, 69(2): 88-112.). At present, the molecular mechanism and intervention targets of occurrence and development thereof are unknown, and there is still no effective treatment for obesity. Although there are a variety of drugs that act on different targets for obesity treatment, for example, appetite suppressant drugs such as phentermine, amfebutamone, and pramlintide; nutrient absorption intervention drugs such as orlistat and cetilistat; and nutrition metabolism promoting drugs such as beloranib (Kheniser et al. The Journal of Clinical Endocrinology & Metabolism, 2021; Idris et al. Diabetes, Obesity and Metabolism, 2009, 11(2): 79-88; Velazquez et al. Annals of the New York Academy of Sciences, 2018, 1411(1): 106-119.). However, because these drugs will induce side effects such as hypertension, emotional disorders, and cardiomyopathy, and bodyweight rebound is likely to occur after drug withdrawal (D'Antongiovanni et al. Frontiers in Pharmacology, 2021, 11: 2415.), they cannot meet the needs of obesity patients yet. Therefore, seeking for safe and effective methods and new drugs for obesity prevention and treatment has become a hot topic of global concern.

5′-Methylthioadenosine (MTA) is an endogenous methionine (Met) metabolite that is widely distributed in the biosphere. Mammalian cells, prokaryotes, fungi, and plant cells can produce a small amount of MTA through different metabolic pathways, and biological functions of MTA are mainly involved in cell proliferation, differentiation, apoptosis and other processes.

Currently, MTA has been found to have potential medicinal value in immunoregulation, cancer control, and neuroprotection, for example:

1) 5′-Methylthioadenosine can inhibit the production of pro-inflammatory genes and cytokines, as well as increase the production of anti-inflammatory cytokines, treating brain autoimmune diseases; in addition, it has a relieving effect on multiple sclerosis and some other autoimmune diseases (Moreno et al. Diss. Abstr. Int., C 2007, 68(1), 111; Ann Neurol 2006; 60: 323-334). Therefore, the pharmaceutical value of MTA for anti-inflammatory, antipyretic and analgesic is disclosed in U.S. Pat. No. 4,454,122 (Bioresearch S.r.I.); The use of MTA for preventing and treating autoimmune diseases such as multiple sclerosis and graft rejection is disclosed in Patent WO2006097547.

2) Use of compounds that inhibit methylthioadenosine phosphorylase (MTAP) in cancer treatment is disclosed in Patent WO2004/074325. In a chemically induced liver cancer model, a decrease in the level of 5′-methylthioadenosine was observed, and it is found that liver preneoplastic lesions and DNA synthesis were inhibited after the administration of exogenous 5′-methylthioadenosine.

3) 5′-Methylthioadenosine is capable of partially inhibiting the increase in astrocyte reactivity due to neuron destruction (Zhongguo Shenjing Kexue Zazhi 1999, 15(4), 289-296; Yike Daxue Xuebao 1999, 26(5), 318-320). Neuroprotective properties of MTA in protectsing cerebral cortical astrocytes and neurons under hypoxia or glucose environment are disclosed in Chinese Patent Publication No. CN 102573854 A.

At present, there is no relevant report on the use of 5′-methylthioadenosine in the preparation of obesity-suppressing drugs or health care products.

### SUMMARY

To overcome the deficiencies of the prior art, an objective of the present disclosure is to provide use of 5′-methylthioadenosine in preparation of obesity-suppressing drugs or health care products.

To achieve the above objective, the present disclosure provides the following technical schemes:

In a first aspect, the present disclosure provides use of 5′-methylthioadenosine in preparation of obesity-suppressing drugs or health care products.

Wherein, the chemical structural formula of the 5′-methylthioadenosine is as follows:

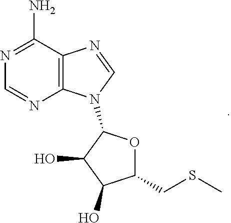 [SMILES: CSC[C@H]1O[C@@H](n2cnc3c(N)ncnc32)[C@H](O)[C@@H]1O]

In some embodiments, the obesity is induced by lipid accumulation in HepG2 cells or high-fat diet.

In a second aspect, the present disclosure provides an obesity-suppressing drug or health care product, where an active ingredient of which is 5′-methylthioadenosine.

The beneficial effects of the present disclosure are:

In the process of studying the suppression of obesity by metabolites of intestinal bacteria, the inventors have unexpectedly found that 5′-methylthioadenosine can significantly suppress obesity induced by high-fat diet in mice, inhibit the lipogenesis of liver and the increases of subcutaneous fats and visceral fats, and improve oral glucose tolerance and insulin sensitivity in mice.

It is demonstrated from animal experiments that the 5′-methylthioadenosine has a significant suppression effect on obesity and has an excellent application prospect in the preparation of the obesity-suppressing drugs or health care products. In the present disclosure, the 5′-methylthioadenosine is compared with adenosine, and the experimental results show that the 5′-methylthioadenosine has a better obesity-suppressing effect.

### BRIEF DESCRIPTION OF THE DRAWINGS

FIG. 1A-FIG. 1F show a microbial synthesis pathway and isotope synthetic products of MTA, where FIG. 1A shows a pathway for synthesizing MTA by Bifidobacterium longum (B. longum) using methionine; FIG. 1B shows the ¹³C labeling of methionine; FIG. 1C shows the mass spectrometry (MS) information of the product MTA synthesized by B. longum using isotope-labeled methionine, where the amount of synthetic MTA is proportional to the methionine concentration; FIG. 1D shows a liquid chromatogram; FIG. 1E is the drawings of partial enlargement of FIG. 1C; FIG. 1F is the drawings of partial enlargement of FIG. 1D;

FIG. 2A-FIG. 2D show the suppression effect of 5′-methylthioadenosine and adenosine on high-fat diet-induced obesity in C57/BL mice; FIG. 2A shows the body weight of the mice at the time of sacrifice; FIG. 2B shows the liver weight of the mice; FIG. 2C shows the subcutaneous fat weight of the mice; FIG. 2D shows the visceral fat weight of the mice.

FIG. 3 shows the inhibition of that 5′-methylthioadenosine suppresses lipogenesis in HepG2 hepatoma cells;

FIG. 4A-FIG. 4I show the suppression effect of 5′-methylthioadenosine on high-fat diet-induced obesity in C57/BL mice, where FIG. 4A shows an experimental design; FIG. 4B shows a growth curve of body weight; FIG. 4C shows body weight gain; FIG. 4D shows liver weight;

FIG. 4E shows hematoxylin-eosin (H&E) staining of liver tissue; FIG. 4F shows subcutaneous fat weight; FIG. 4G shows visceral fat weight; FIG. 4H shows oral glucose tolerance test (OGTT); FIG. 4I shows insulin tolerance test (ITT);

FIG. 5A-FIG. 5J show the suppression effect of MTA on high-fat diet-induced obesity in C57/BL mice, where FIG. 5A shows a growth curve of body weight; FIG. 5B shows body weight gain; FIG. 5C shows liver weight; FIG. 5D shows subcutaneous fat weight; FIG. 5E shows testicular fat weight; FIG. 5F shows perirenal fat weight; FIG. 5G shows OGTT; FIG. 5H shows area under curve (AUC) of OGTT; FIG. 5I shows ITT; FIG. 5J shows AUC of ITT.

In all figures, Chow represents the low-fat diet group, HFD represents the high-fat diet group, MTA represents the 5′-methylthioadenosine-treated group, and Adenosine represents the adenosine-treated group.

### DETAILED DESCRIPTION OF THE EMBODIMENTS

The present disclosure will be further described below in conjunction with the example. It should be noted that the following descriptions are only intended to explain the present disclosure and do not limit the content thereof.

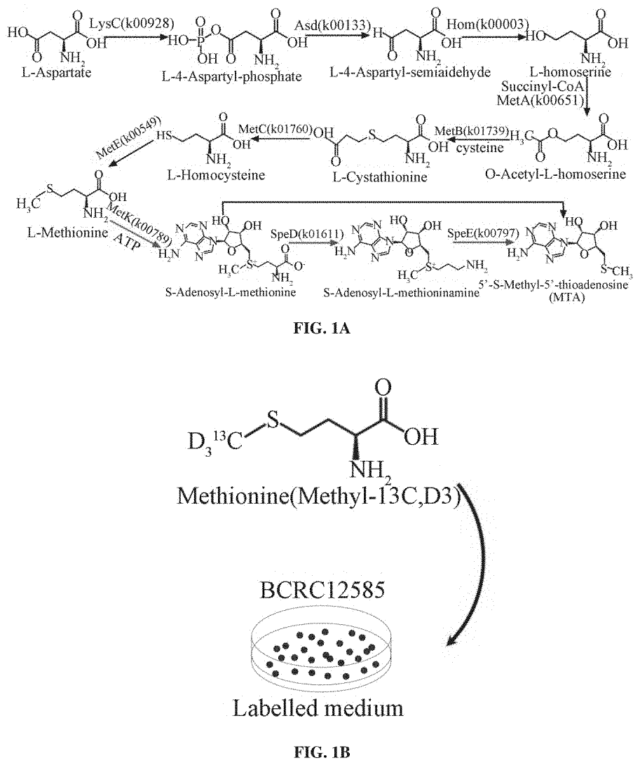

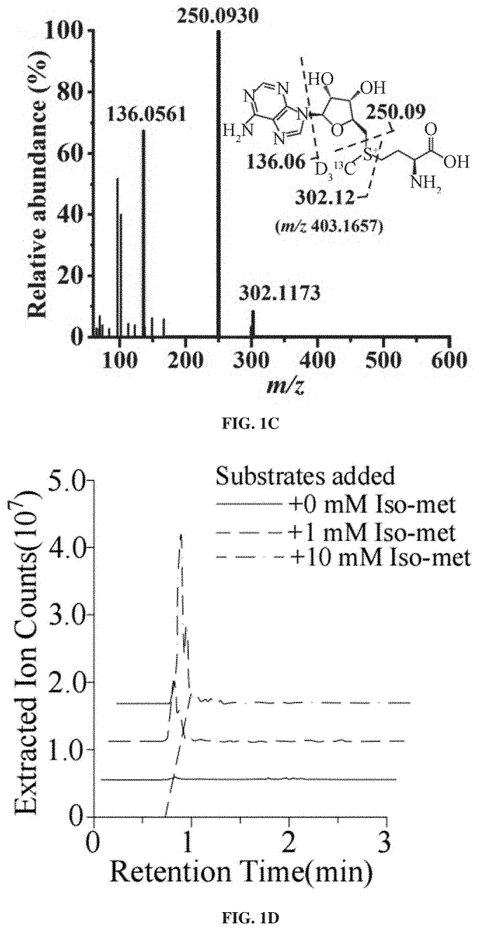

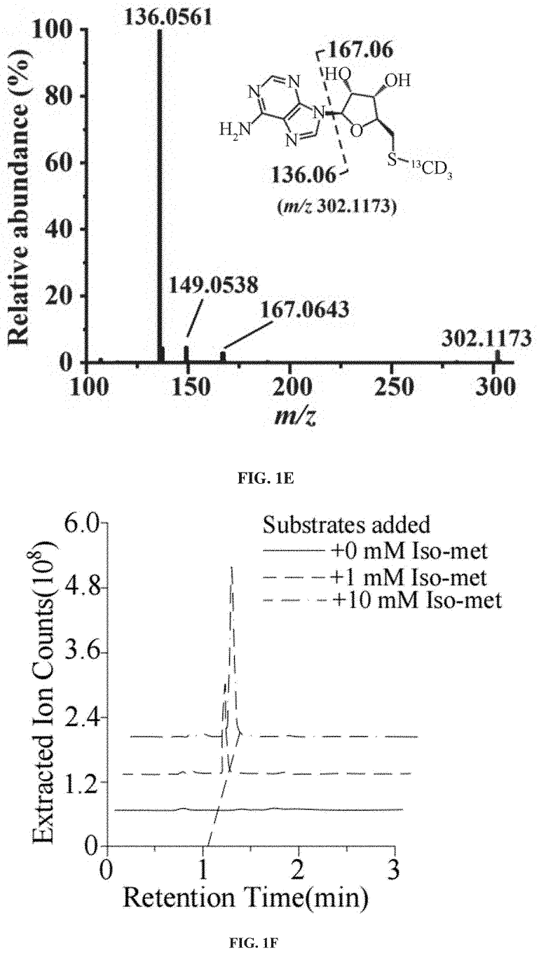

### Synthesis of MTA (FIG. 1A-FIG. 1F)

5′-Methylthioadenosine (MTA) was produced by fermentation of human intestinal bacteria (B. longum). B. longum was inoculated in an anaerobic fermenter; the GMM medium with a certain concentration of methionine was used; the GMM medium cultured for 24 h was lyophilized and extracted with 70% (v/v) ethanol aqueous solution. The supernatant was concentrated and centrifuged at a high speed of 12,000 r/min, and 5′-methylthioadenosine monomer with a purity of >98% was obtained by separation and purification using a preparative liquid chromatography system.

The microbial synthesis pathway and isotope synthetic products are shown in FIG. 1A-FIG. 1F.

1. Comparison of MTA and Adenosine

Experimental animals: 8-week-old C57BL/6 mice;

Diets and drugs: low-fat diet (10% kcal as fat, product #D12450J, Research Diets), high-fat diet (60% kcal as fat, product #D12492, Research Diets), MTA, and adenosine.

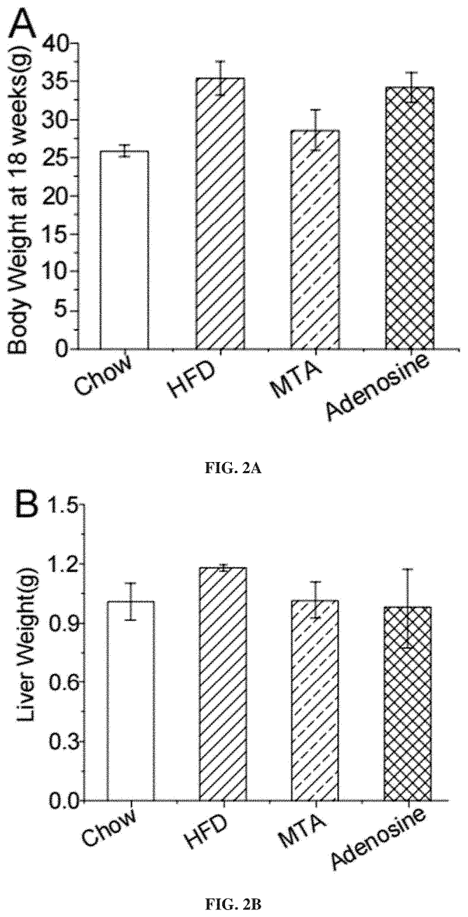

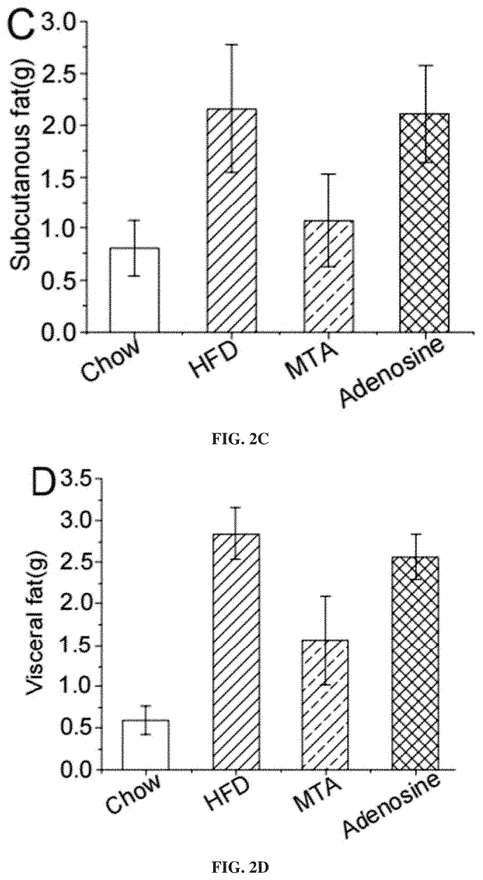

Comparison of the difference in suppressing obesity between MTA and adenosine: At a tube-feeding concentration of 100 mg/kg/day, although adenosine was shown to inhibit the body weight and liver weight gains and fat accumulation in mice, the obesity-suppressing effect of MTA was significantly better than that of adenosine (FIG. 2A-FIG. 2D).

2. Suppression of Lipogenesis in HepG2 Hepatoma Cells by 5′-Methylthioadenosine

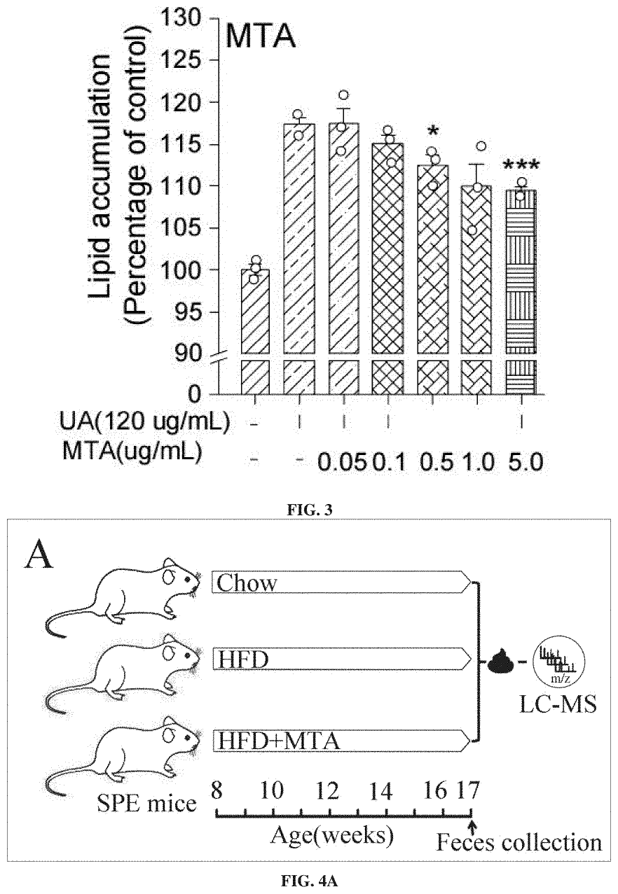

HepG2 hepatoma cells were induced to synthesize fat by uric acid (120 μg/mL). After 24 h, the cells were divided into two groups for testing. The cells of model group continued to be induced with uric acid (120 μg/mL) for 24 h. The cells of drug-treated group were treated with uric acid (120 μg/mL)+5′-methylthioadenosine (0.05, 0.1, 0.5, 1.0, and 5.0 μg/mL) for 24 h, then the amount of synthetic fat by cells was detected by oil red staining, and it was found that 5′-methylthioadenosine was capable of significantly inhibiting uric acid-induced lipogenesis in HepG2 cells with a concentration gradient effect (FIG. 3).

3. Suppression of High-Fat Diet-Induced Obesity by 5′-Methylthioadenosine

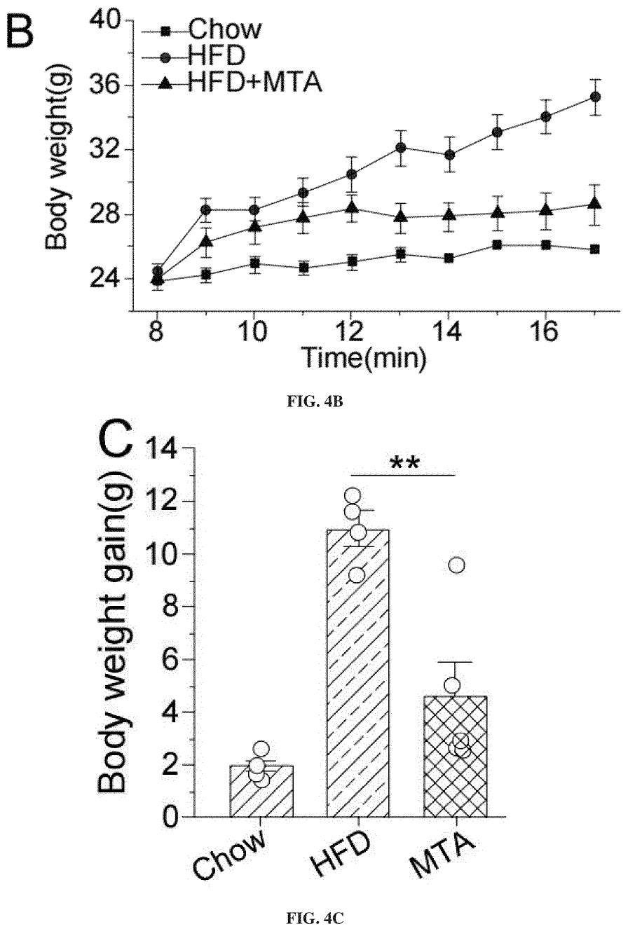

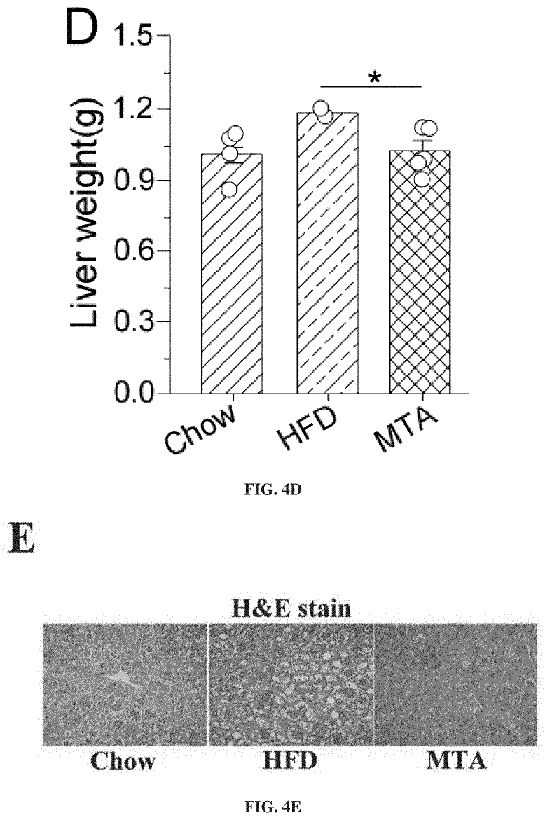

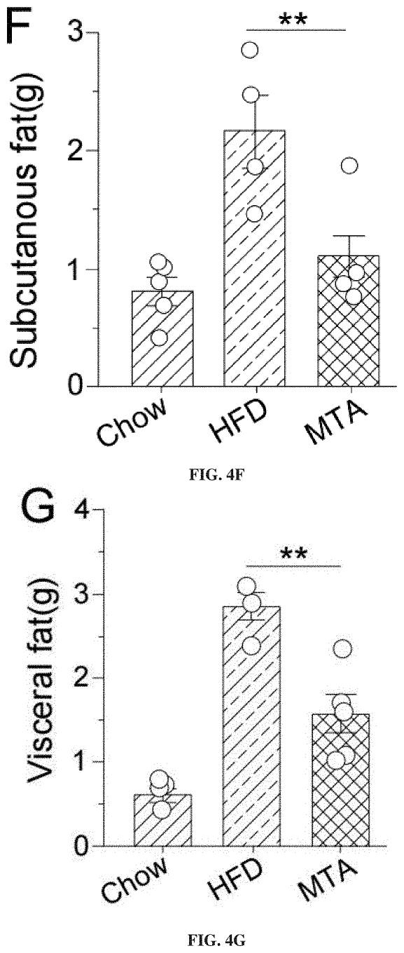

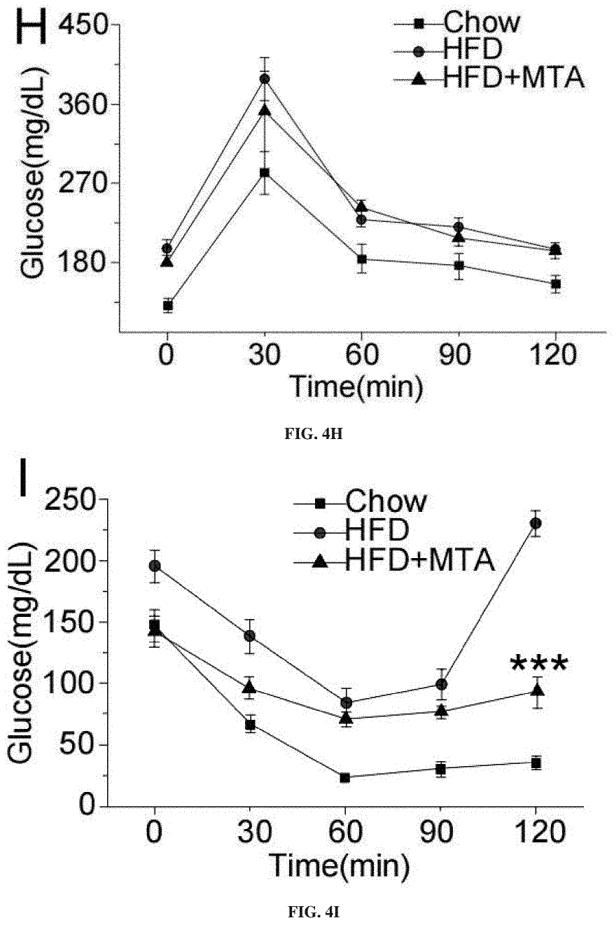

Eight-week-old C57BL/6 mice were divided into three groups (a control group, a model group, and a drug-treated group) (FIG. 4A). The mice of control group were fed with a low-fat diet, and the mice of model group were fed with a high-fat diet ad libitum. The mice of drug-treated group were fed with high-fat diet+5′-methylthioadenosine (100 mg/kg/day) by gavage for 9 weeks, during which the body weight of the mice was recorded, and OGTT and ITT thereof were conducted. It was found that 5′-methylthioadenosine was capable of significantly suppressing high-fat diet-induced obesity (FIG. 4B), and improving oral glucose tolerance and insulin tolerance in mice (FIG. 4H and FIG. 4I). After sacrifice, the liver, subcutaneous fat, and visceral fat of the mice were weighed, and it was found that 5′-methylthioadenosine was capable of suppressing the liver lipogenesis and the gains of subcutaneous fats and visceral fats (FIG. 4C to FIG. 4G).

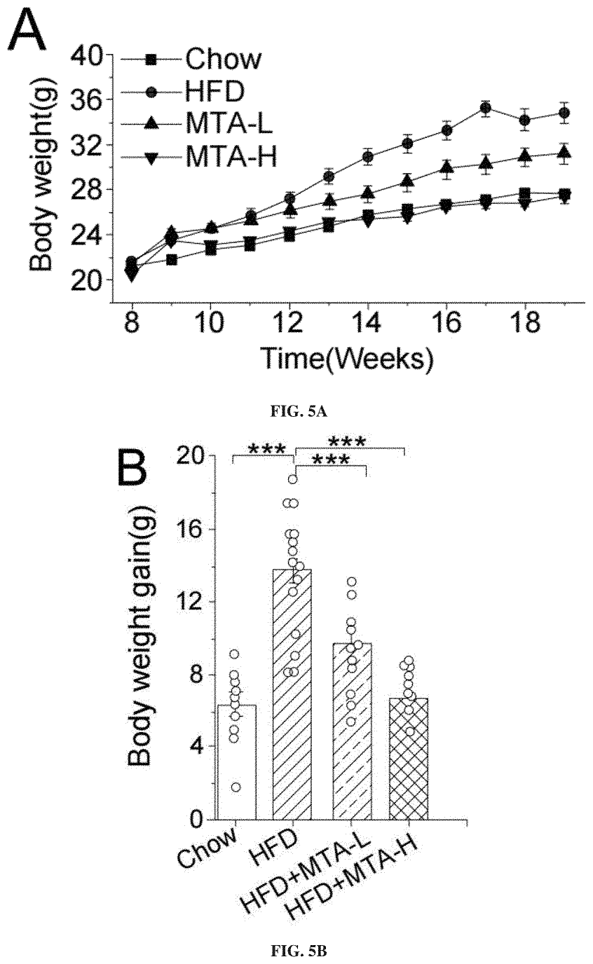

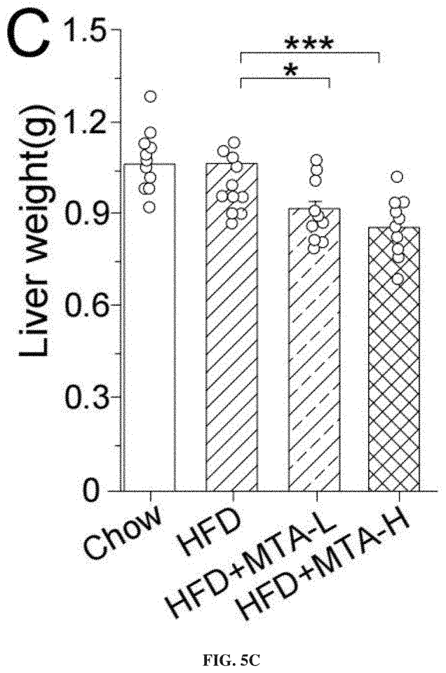

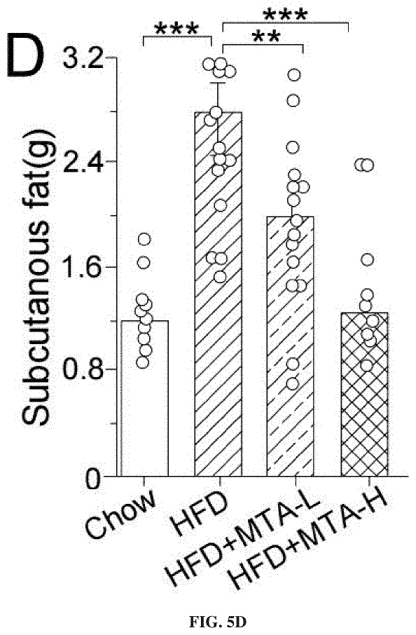

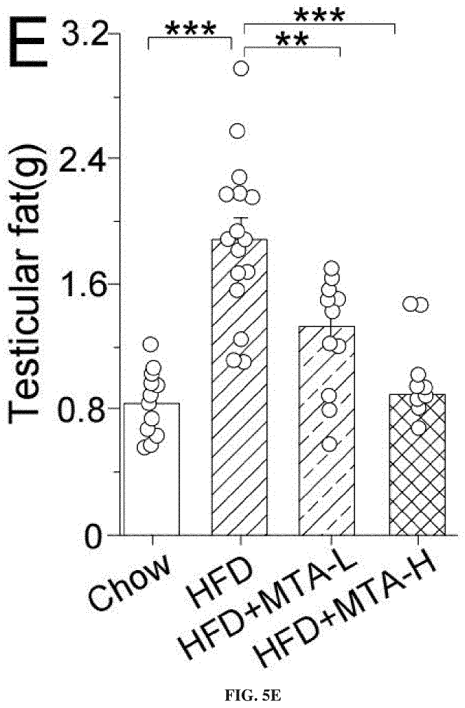

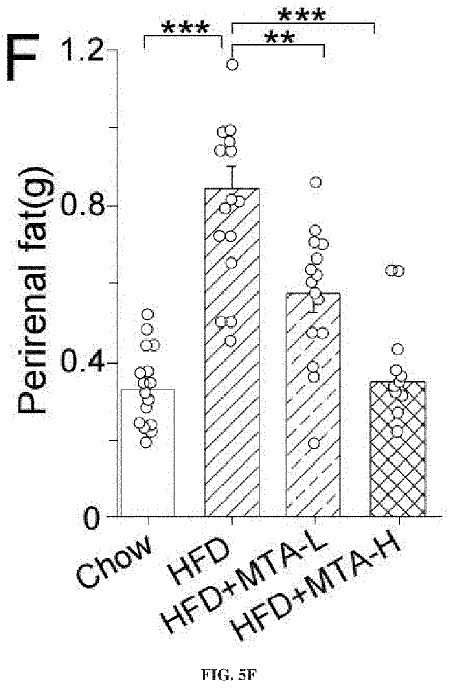

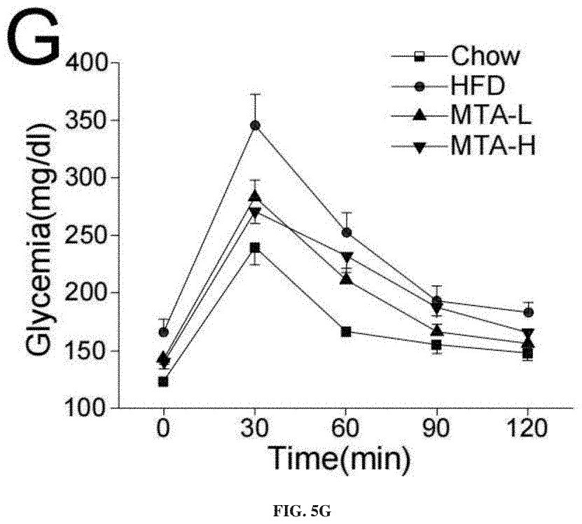

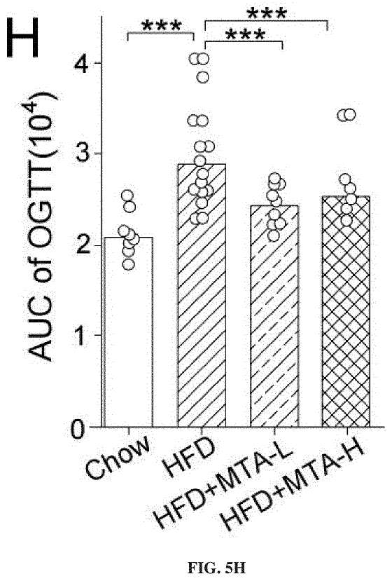

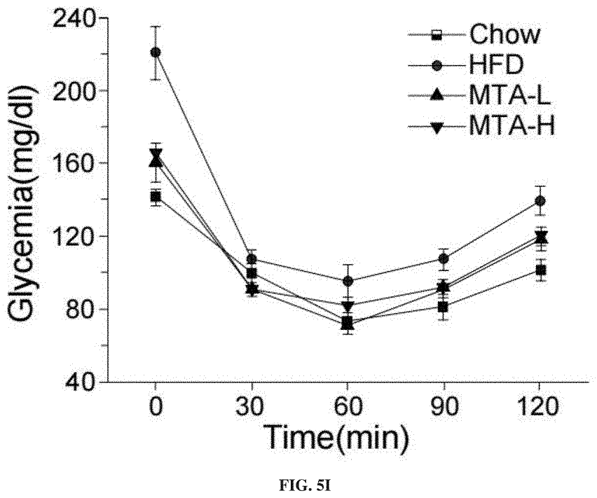

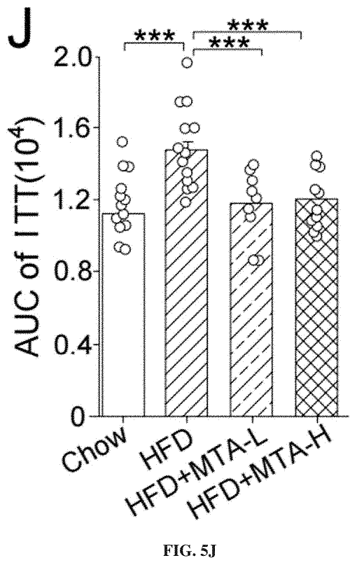

Eight-week-old C57BL/6 mice were divided into three groups (a control group, a model group, and a drug-treated group). The mice of control group were fed with a low-fat diet, the mice of model group were fed with a high-fat diet, and the mice of drug-treated group were fed with high-fat diet+5′-methylthioadenosine (50 and 100 mg/kg/day) by gavage for 11 weeks, during which the body weight of the mice was recorded, and OGTT and ITT thereof were conducted. It was found that 5′-methylthioadenosine was capable of significantly suppressing high-fat diet-induced obesity, with a concentration gradient effect (FIG. 5A and FIG. 5B). Meanwhile, it was capable of improving oral glucose tolerance and insulin tolerance in mice (FIG. 5G to FIG. 5J). After sacrifice, the liver, subcutaneous fat, perirenal fat, and testicular fat of the mice were weighed, and it was found that 5′-methylthioadenosine was capable of suppressing the liver lipogenesis and the gains of subcutaneous fats and visceral fats, with a concentration gradient effect (FIG. 5C to FIG. 5F).

Although the specific embodiments of the present disclosure are described above, they do not intended to limit the protection scope of the present disclosure. On the basis of the technical schemes of the present disclosure, various modifications or variations made without creative efforts by those skilled in the art still fall within the protection scope of the present disclosure.

1. A method for treating obesity, comprising administering drugs or health care products comprising 5′-methylthioadenosine to a subject in need thereof.

2. An obesity-suppressing drug or health care product, comprising 5′-methylthioadenosine as an active ingredient.
